(19) United States
(12) Patent Application Publication
(10) Pub. No.: US 2019/0011472 A1
(43) Pub. Date: Jan. 10, 2019

(54) SAMPLING DEVICE

(71) Applicant: Zeta Biopharma GmbH, Lieboch (AT)

(72) Inventor: Erwin TRUMMER, St. Stefan im Rosental (AT)

(73) Assignee: Zeta Biopharma GmbH, Lieboch (AT)

(21) Appl. No.: 16/028,053

(22) Filed: Jul. 5, 2018

(30) Foreign Application Priority Data
Jul. 6, 2017 (AT) A 50563/2017

(51) Int. Cl. G01N 35/10; C12M 1/26; G01N 1/08

(52) U.S. Cl. CPC G01N 35/10; C12M 33/04; B01L 3/50; G01N 1/08; G01N 2001/085; C12M 33/12

(57) ABSTRACT

A sampling device (1) for filling a sample vessel (4) with sample material from a material container, wherein the sampling device (1) comprises a first locking unit (2), a sterilization unit and a sampling chamber (3), wherein the sampling chamber (3) has an internal chamber space (5), a manipulation unit (6), an opening unit (7) arranged within the internal chamber space (5) as well as a coupling unit (8) arranged within the internal chamber space (5), the first locking unit (2) is arranged between the material container and the coupling unit (8), and the sterilization unit is connected to the internal chamber space (5) and configured to sterilize the internal chamber space (5), wherein the manipulation unit (6) is configured to accommodate the sample vessel (4) at least in part within the internal chamber space (5), movable between the opening unit (7) and the coupling unit (8), the opening unit (7) is configured to open and close the sample vessel (4), and the coupling unit (8) is configured to be coupleable to the sample vessel (4).

This application claims benefit of Serial No A 50563/2017, filed 6 Jul. 2017 in Austria and which application is incorporated herein by reference. To the extent appropriate, a claim of priority is made to the above disclosed application.

### BACKGROUND OF THE INVENTION

The invention relates to a sampling device for filling a sample vessel with sample material from a material container.

The invention further relates to a method for filling a sample vessel with sample material from a material container using a sampling device.

In the fields of the biopharmaceutical and the food industry, high standards are applied in regard to production, storage and transportation of products. Legal provisions in these fields regulate, among others, the sterility of production processes, processing processes and the application of the products as well as the performance of product inspections. Within the scope of production, hence, due to the requirements of quality assurance and the legal provisions, it is necessary to continuously inspect products in regard to contamination by germs and bacteria. In this way there will be ensured that the quality of production will meet the legal standards.

Biopharmaceutical products or those of the food industry are usually kept in large aseptic material containers during the production process, in which they are transported, temporarily stored or processed. According to prior art, sampling from the material containers is realized by opening these, removing a part of the product from the material container using a device as sterile as possible, and filling it into a sample vessel that has been sterilized beforehand. In particular in course of sampling materials containing pieces such as, e.g., fruit preparations having large fruit pieces in food industry, it is necessary to open the material container as the fruit pieces can be sucked from the material container only with difficulty. This form of sampling meets the legal provisions and allows for the validation of product batches having no contamination.

Sampling devices according to prior art, which rely on this principle, however, have the disadvantage that the aseptic material container has to be opened when removing sample material. In this way, there is developed a substantial risk for contamination of the sample material during sampling. This may lead thereto that the sample is contaminated unnoticed, even though the product in the material container does not exhibit any contamination. As a consequence of a positive test in regard to contamination of the sample material, the actually contamination-fee product in the material container will be discarded. This leads to substantial economic damage for the producer, as several tons of the product may be contained in such a material container. In addition, there is given the risk of unnoticed contamination of the product in the material container itself in the course of sampling, which may have the consequence of the disadvantage of a contamination entrainment within the production facility.

### SUMMARY OF THE INVENTION

It is the task of the present invention to provide a sampling device and a method, which avoid the disadvantages mentioned above.

According to the invention the present task is solved by the sampling device comprising a first locking unit, a sterilization unit and a sampling chamber, wherein the sampling chamber has an internal chamber space, a manipulation unit, an opening unit arranged within the internal chamber space as well as coupling unit arranged within the internal chamber space, the locking unit is arranged between the material container and the coupling unit, and the sterilization unit is connected to the internal chamber space and configured to sterilize the internal chamber space, wherein the manipulation unit is configured to accommodate the sample vessel at least in part within the internal chamber space, movable between the opening unit and the coupling unit, wherein the opening unit is configured to open and close the sample vessel and wherein the coupling unit is configured to be coupleable to the sample vessel.

The present task is furthermore solved by a method for filling a sample vessel with sample material from a material container using a sampling device with the following method steps:
a) flooding an internal chamber space of the sampling device with hot steam of a temperature of at least 120° C.;
b) reducing the temperature of the hot steam to below 120° C.;
c) introducing the sample vessel into the internal chamber space;
d) opening the sample vessel;
e) connecting the sample vessel to the material container;
f) filling the sample vessel with sample material from the material container;
g) closing the sample vessel preferably under hot steam;
h) removing the sample vessel from the internal chamber space.

The embodiment according to the invention of the sampling device provides for a first locking unit, a sterilization unit and a sampling chamber. The sampling chamber itself has a manipulation unit and an internal chamber space, into which there is introduced a closed and sterilized sample vessel for carrying out aseptic sampling. In the internal chamber space there are situated further an opening unit and a coupling unit, which is connected to the first locking unit. The first locking unit is connected to the material container and may, for example, be provided in the form of a valve. The sterilization unit, which may be provided in the form of a steam generator, is connected to the internal chamber space and sterilizes the internal chamber space and the further components of the sampling device, which are situated in the internal chamber space, in the course of sampling. The sample vessel is opened by means of the opening unit, it is moved from the opening unit to the coupling unit by means of the manipulation unit and it is subsequently connected to the coupling unit. Then the first locking unit is opened and a defined amount of sample material from the sample container is filled into the sample vessel. Subsequently, the sample vessel is separated from the coupling unit, and it is again moved to the opening unit by means of the manipulation unit. By means of the opening unit, the sample vessel is subsequently closed again, and then the sample vessel is removed from the sampling chamber.

Due to the design of the sampling device according to the invention, there is obtained the advantage that there is provided a completely aseptic and closed environment for sampling. It is especially advantageous that before opening the first locking unit the internal chamber space of the sampling chamber is sterilized by the sterilization unit, whereby any contaminations, which have been introduced into the internal chamber space with the sample vessel, will be removed. This provides for a completely aseptic sampling and prevents possible contaminations of the sample material or of the material container.

It is especially advantageous that the process of sterilization by the sterilization unit is performed completely within the internal chamber space. This minimizes the risk for injuries for working staff, which is usually associated with the application of hot steam or chemicals for sterilization.

### BRIEF DESCRIPTION OF THE DRAWINGS

Advantageous embodiments of the sampling device according to the invention and of the method as well as alternative embodiment variants will be explained in greater detail below by way of the figures.

FIG. 1 shows a section through a sampling device according to the invention having a sample vessel partially accommodated within the sampling device in a side view.

FIG. 2 shows a section through a sampling device according to the invention having a sample vessel partially accommodated within the sample device and a sampling line in a side view.

### DETAILED DESCRIPTION OF THE PREFERRED EMBODIMENT

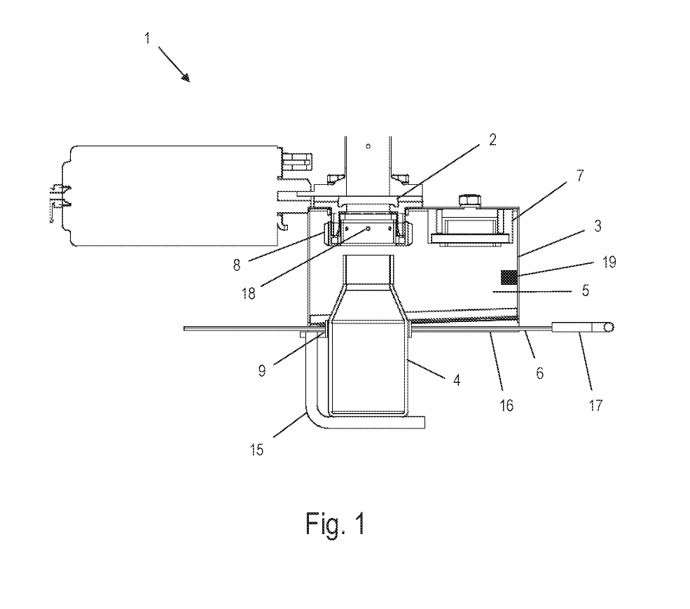

FIG. 1 shows as sectional view of a sampling device 1 according to the invention having a first locking unit 2, a sterilization unit, which is not depicted in FIG. 1, and a sampling chamber 3. In the sampling device 1 depicted in FIG. 1, a sample vessel 4 is partially accommodated within the sampling chamber 3. The sampling chamber 3 has an internal chamber space 5, a manipulation unit 6, an opening unit 7 arranged within the internal chamber space 5 as well as a coupling unit 8, which is also situated within the internal chamber space 5. The first locking unit 2 is arranged between a material container, which is not depicted in FIG. 1, and the coupling unit 8 and provides for or prevents, respectively, a passage of sample material from the material container into the sample vessel 4. The opening unit 7 and the coupling unit 8 are arranged next to each other in the embodiment of the sampling device 1 depicted in FIG. 1. The first locking unit 2, for example, is configured as an electrically, pneumatically or mechanically actuated valve. Further embodiment variants of the first locking unit 2 become obvious for those skilled in the art from this exemplary reference.

The sterilization unit is configured to sterilize the internal chamber space 5, wherein the sterilization unit is connected to the internal chamber space 5. In this process, all further elements situated therein will be sterilized. In the preferred embodiment variant of the sampling device 1, the sterilization unit is configured as a steam generator, which floods the internal chamber space 5 with hot steam in the course of the sterilization process. This has the advantage that steam generators are very common on the market, thus being readily available. Furthermore, these do not need any potentially hazardous chemicals for the sterilization process. Especially preferred is that the steam generator generates steam of a temperature of at least 120° C. This has the advantage that in this way there is obtained a very good result of the sterilization process. In an alternative embodiment variant, the sterilization unit sterilizes the internal chamber space 5, for example, using chemical methods. Further embodiment variants of the sterilization unit will become obvious for those skilled in the art from this exemplary reference.

In a preferred embodiment variant the sampling device 1 has a first temperature sensor 19, which is arranged within the internal chamber space 5. This provides the advantage that in this way it is made possible to determine the steam temperature within the internal chamber space 5, thus preventing it from dropping below a determined predefined value. It is especially advantageous that by way of this examination it will be ensured that the sterilization process is carried out especially effectively in each sampling.

The manipulation unit 6 accommodates the sample vessel 4 at least in part within the internal chamber space 5, movable between the opening unit 7 and the coupling unit 8. The manipulation unit 6 is configured in the preferred embodiment variant of the manipulation unit 6 that is depicted in FIG. 1 as a slidable boundary surface of the sampling chamber 3, which has an opening 9 adapted to a cross-section of the sample vessel 4. The manipulation unit 6 is, for example, embodied as a slidable metal sheet forming the boundary surface of the sampling chamber 3. In order to accommodate the sample vessel 4 in the sampling chamber 3, the manipulation unit 6 has an opening 9 adapted to the cross-section of the sample vessel 4, into which the sample vessel 4 is introduced. Upon flooding the internal chamber space 5 with hot steam by the sterilization unit, the sample vessel 4 seals the opening 9 of the manipulation unit 6 and, hence, the internal chamber space 5. In this way, the steam is retained within the internal chamber space 5, whereby there is obtained the advantage that no special precautions for handling the sampling device 1 according to the invention are required.

In the course of sampling using the sampling device 1 according to the invention, the sample vessel 4 is introduced into the opening 9 of the manipulation unit 6. This is positioned such that the sample vessel 4 may be connected to the opening unit 7. The sample vessel 4 itself may, for example, be a laboratory flask and has a closure. The opening unit 7 is configured to open and close the sample vessel 4. In the meantime, the opening unit 7 engages the closure of the sample vessel 4 and retains this at the opening unit 7 upon opening of the sample vessel 4. Thereafter, the sample vessel 4 is moved to the position of the coupling unit 8 by means of the manipulation unit 6. The coupling unit 8 is configured to be coupleable to the sample vessel 4, wherein the opened sample vessel 4 is, for example, screwed into the coupling unit 8. In alternative embodiment variants there may be provided further coupling methods such as, e.g., bayonet closures or clamping fittings. In this way, the sample vessel 4 is connected to the material container. Subsequently to coupling of the sample vessel 4 to the coupling unit 8, the first locking unit 2 is opened and an amount of sample material from the material container is filled into the sample vessel 4. Then the first locking unit 2 is closed again, and the coupling of the sample vessel 4 to the coupling unit 8 is released. As a consequence, the sample vessel 4 is moved again to the position of the opening unit 7 by means of the manipulation unit 6 and closed by the opening unit 7 by means of the closure having previously remained at the opening unit 7. Finally, the sample vessel 4 is removed from the manipulation unit 6 and, hence, from the internal chamber space 5. The entire process of sampling described is carried out under sterile conditions. For this purpose, in the preferred embodiment variant the sterilization unit generates a hot steam atmosphere within the internal chamber space 5. Opening the sample vessel 4, coupling to the coupling unit 8, the filling process and closing the sample vessel 4 are all carried out in the hot steam atmosphere. In this way, there is obtained the advantage that any germs and bacteria present will be killed, with the sample material remaining free of germs. Furthermore, due to this process there is no risk for contamination of the content of the material container.

In a preferred embodiment variant of the coupling unit 8, the coupling unit 8 has sterilization openings 18, which are connected to the sterilization unit. This has the advantage that the sample vessel 4 may be additionally steam-treated in that area, in which the coupling unit 8 engages in the course of coupling. In this way, there is obtained the advantage that any possibly existent contaminations are removed in this area.

The sampling chamber 3 in a preferred embodiment variant has connecting elements 16, wherein the manipulation unit 6 is accommodated slidable within the connecting elements 16. In the embodiment of the manipulation unit 6 as an iron sheet as previously described, the connecting elements 16 are, for example, embodied as guide grooves arranged on the sampling chamber 3, in which the manipulation unit 6 may be slidably moved for moving the sample vessel 4. This has the advantage that the connecting elements 16 provide a safe guiding for the manipulation unit 6, whereby there is minimized the risk that steam exits from the internal chamber space 5 during the sterilization process.

In an especially preferred embodiment variant, the manipulation unit 6 has a handle 17. This has the advantage that the handle 17 ensures easy handling of the manipulation unit 6. The handle 17 further has the advantage that the manipulation unit 6 may also be moved without any additional safety equipment if the manipulation unit 6 is heated due to the sterilization process using hot steam.

The first locking unit 2 of the sampling device 1 has in a preferred embodiment variant a passage cross-section of at least 30 mm. The coupling unit 8 is further configured to couple a sample vessel 4, which has an opening area having a diameter of at least 30 mm. This has the advantage that the sampling device 1 may also be used for inhomogeneous material such as, for example, fruit preparations containing whole fruit pieces.

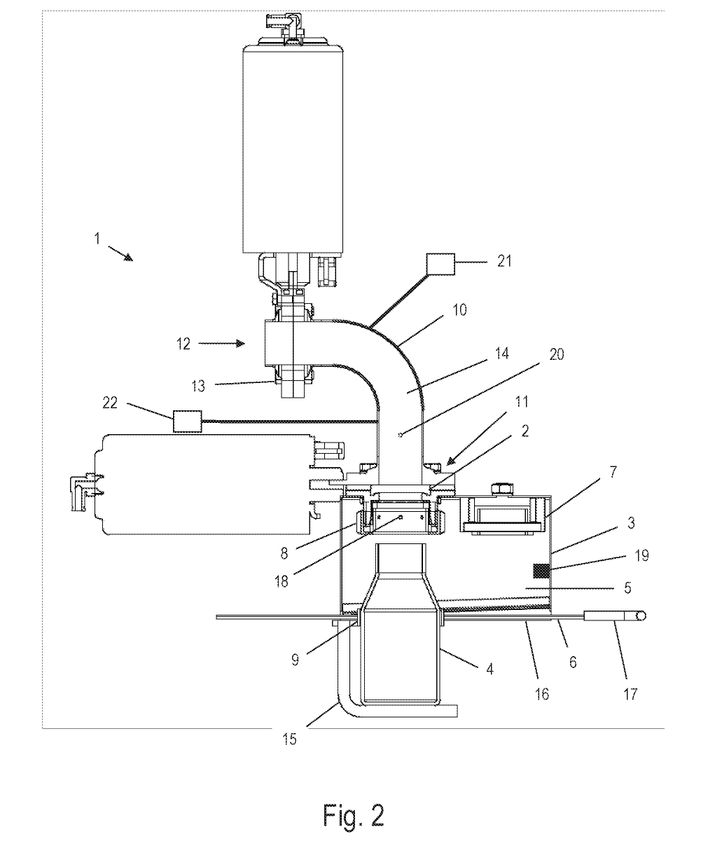

FIG. 2 shows a section through a sampling device 1 according to the invention in a side view in the preferred embodiment variant having an additional sampling line 10, which has an internal line space 14. The sampling line 10 further has a first end 11, at which the first locking unit 2 is arranged, and a second end 12, at which a second locking unit 13 is arranged. The second end 12 of the sampling line 10 is connected to the material container, which is not depicted in FIG. 2. The first locking unit 2 and the second locking unit 13 are configured to lock the sampling line 10. The first locking unit 2 and the second locking unit 13 may, among others, be embodied as electrically, pneumatically or mechanically actuated valves. Further embodiment variants of the first locking unit 2 and of the second locking unit 13 will become obvious for those skilled in the art from this exemplary reference.

The arrangement of the sampling line 10, which may be locked by the first locking unit 2 and the second locking unit 13 at the first end 11 and at the second end 12, between the material container and the sampling chamber 3 has the advantage that the sampling line 10 is filled with a defined amount of sample material during sampling. For this purpose, the internal line space 14 has a volume, which determines a volume of sample material for filling the sample vessel 4. This has the advantage that the amount of sample material removed from the material container is identical in each sampling. In the course of sampling, firstly the second locking unit 13 is opened and then the first locking unit 2 is kept closed. The sampling line 10, as a consequence, fills with the sample material from the material container. Then the second locking unit 13 is closed, and the first locking unit 2 is opened in order to fill the sample material into the sample vessel 4. This embodiment of the sampling line 10 has the additional advantage that due to the sequential opening of the second locking unit 13 and the first locking unit 2 there is introduced an additional safety barrier, which further reduces the risk of contamination of the material container.

The sampling line 10 depicted in FIG. 2 has the internal line space 14, in which the sampling material is transported. In the preferred embodiment variant of the sampling device 1, the sterilization unit is connected to the internal line space 14, wherein the sterilization unit sterilizes the internal line space 14. This arrangement has the advantage that the sterilization unit is used to sterilize the internal line space 14 of the sampling line 10 as well as the internal chamber space 5 of the sampling chamber 3 if the second locking unit 13 is opened. In an alternative embodiment variant the sterilization unit is connected to the internal chamber space 5 as well as the internal line space 14.

In a preferred embodiment variant the sampling device 1 has a second temperature sensor 20, which is arranged within the internal line space 14. This provides the advantage that in this way it is made possible to determine the steam temperature within the internal line space 14 and, hence, prevent this from dropping below a determined predefined value. It is especially advantageous that because of this examination there will be ensured that the sterilization process is carried out especially effectively in each sampling. In combination with the first temperature sensor 19, which determines the temperature within the internal chamber space 5, there is ensured an especially continuous examination of the steam temperature within the sampling device 1.

The sampling line 10, the first locking unit 2 and the second locking unit 13 have in the preferred embodiment variation of the sampling device 1 a passage cross-section of at least 30 mm. The coupling unit 8 is further configured to couple a sample vessel 4, which has an opening area having a diameter of at least 30 mm. This has the advantage that the sampling device 1 may also be used for inhomogeneous material such as, for example, fruit preparations containing fruit pieces.

As already described above, the coupling unit 8 has in a preferred embodiment variant sterilization openings 18, which are connected to the sterilization unit. Also in the embodiment variation of the sampling device 1 according to FIG. 2 this has the advantage that the sample vessel 4 may be selectively steam-treated in the course of coupling in that area, in which the coupling unit 8 engages. In this way there is obtained the advantage that any possibly present contaminations in this area are removed and that in addition it is prevented that contaminations enter the internal line space 14 of the sampling line 10 in the course of coupling. The sterilization openings 18 may in an alternative embodiment variant also be connected to the sterilization unit via the internal line space 14.

Furthermore, the sampling device 1 has in a preferred embodiment variant a negative pressure generating unit 22, which is connected to the internal line space 14. In order to support transport of the sample material from the material container into the internal line space 14, the negative pressure generating unit 22 may be provided in order to generate a negative pressure within the internal line space 14. The negative pressure generating unit 22 may, for example, be embodied as a vacuum pump. The negative pressure generating unit 22 provides the advantage that due to the negative pressure generated the transport of the sample material from the material container into the sampling line 10 is realized especially rapidly and that the internal line space 14 is filled as completely as possible with sample material. Advantageously, the negative pressure generated by the negative pressure generating unit 22 is only slightly below the pressure existent in the material container. In this way there is ensured that any possibly present inhomogeneities in the sample material such as, for example, fruit pieces in a yoghurt, will not be destroyed. In an alternative embodiment variant the negative pressure generating unit 22 may be connected also to the internal chamber space 5 due to the only small pressure difference being required and it may also generate a negative pressure within the internal chamber space 5, whereby transferring the sample material from the internal line space 14 into the sample vessel 4 will be additionally supported.

In a further preferred embodiment variant the sampling device 1 has an inert gas purging unit 21, which is connected to the internal line space 14. The inert gas purging unit 21 generates a positive pressure within the internal line space 14 in relation to the ambient pressure present around the sampling device 1, whereby also the sample material present within the internal line space 14 is pressurized. Due to this positive pressure, the transport of the sample material from the sampling line 10 into the sample vessel 4 is supported. Hereby, it is especially advantageous that in this way the risk is reduced that residues of sample material will remain within the sampling line 10 during sampling. As an inert gas, there may be used, for example, nitrogen. Advantageously, there is generated a pressure by the inert gas purging unit 21, which is only slightly above ambient pressure present around the sampling device 1. In this way, analogously to the negative pressure generating unit 22, there is ensured that any inhomogeneities possibly present in the sample material such as, for example, fruit pieces in a yoghurt, will not be destroyed.

As depicted in FIG. 1 and FIG. 2, the sampling chamber 3 in the preferred embodiment variant of the sampling device 1 has a holder 15 arranged underneath the manipulation unit 6 in operational position. The holder 15 secures the sample vessel 4 in the manipulation unit 6, whereby there is obtained the advantage that the sample vessel 4 cannot slide out of the opening 9 of the manipulation unit 6. Especially advantageously it will be ensured that no hot steam may exit from the internal chamber space 5 and that no contamination can enter the internal chamber space 5.

In an especially preferred embodiment variant of the sampling device 1 the holder 15 is arranged underneath the coupling unit 8 in the operational position. This offers the advantage that in the case of an inadvertent separation of the coupling between the coupling unit 8 and the sample vessel 4 during sampling no sample material may exit from the sampling chamber 3. In this way, the sample material remains in a sterile environment.

When sampling using the sampling device 1 according to FIG. 2, the sample device 2 has the following operational sequence: Firstly, the internal line space 14 and the internal chamber space 5 are sterilized using the sterilization unit. In the preferred embodiment variant of the sampling device 1 hereby the internal line space 14 and the internal chamber space 5 are flooded with hot steam having a temperature of at least 120° C., with the second locking unit 13 being closed and the first locking unit 2 being opened, by means of which any germs present in the sampling device 1 will be killed. Then the amount of steam generated by the sterilization unit is reduced, the temperature of steam is reduced to below 120° C., preferably to about 70° C. to 80° C., and the sample vessel 4 that has been sterilized before is introduced into the internal chamber space 5. In this way there is ensured that the working staff operating the sampling device 1 may introduce the sample vessel 4 manually into the internal chamber space 5, without being in danger of experiencing scalding injuries due to too high steam temperatures. Then the sample vessel 4 with the opening unit 7 is opened, wherein a closure of the sample vessel 4 remains on the opening unit 7. Then the sample vessel 4 is coupled to the coupling unit 8, whereby the sample vessel 4 is connected to the material container. During the coupling process the sample vessel 4, in particular in that area, in which the coupling unit 8 engages, is continuously steam-treated through the sterilization openings 18 of the coupling unit 8. In this way there is obtained the advantage that any possibly present contaminations in this area will be removed and in addition it will be prevented that any contaminations possibly introduced with the sample vessel 4 into the internal chamber space 5 enter the internal line space 14 of the sampling line 10 in the course of coupling. Subsequently, the first locking unit 2 is closed, and the second locking unit 13 is opened, whereby the internal line space 14 is filled with sample material from the material container. As a consequence, the second locking unit 13 is again closed, and by opening the first locking unit 2 the sample vessel 4 is filled with the sample material from the sampling line 10. Then the first locking unit 2 is closed, and the sample vessel 4 is closed by the opening unit 7 and the closure having remained thereon. Finally, the sample vessel 4 is removed from the internal chamber space 5.

In a preferred embodiment variant of the method, before filling the internal line space 14 with sample material from the material container, there is generated a small negative pressure within the internal line space 14 by the negative pressure generating unit 22. In this way, there is obtained the advantage that the sample material is transferred especially rapidly into the sampling line 10 and that the internal line space 14 is filled with sample material as completely as possible. Subsequently, there is generated a positive pressure within the internal line space 14 by the inert gas purging unit 21, whereby the sample material is transferred into the sample vessel 4 as completely as possible upon opening the first locking unit 2. In this way, there is obtained the advantage that the sampling line 10 is emptied as free of residues as possible.

The method according to the invention for filling a sample vessel 4 with sample material from the material container has the following method steps: Firstly, the internal chamber space 5 is flooded with hot steam having a temperature of at least 120° C. In this way, the internal chamber space 5 is sterilized. Subsequently, the temperature of the hot steam is reduced to below 120° C. In this way there is obtained the advantage that working staff of the sampling device 1 may manually introduce the sample vessel 4 into the internal chamber space 5, without being in danger of experiencing scalding injuries due to too high steam temperatures or without having to wear protective gear. Upon reduction of the temperature, the sample vessel 4 is then introduced into the internal chamber space 5 and then opened. Subsequently, the sample vessel 4 is connected to the material container and filled with sample material from the material container. Thereafter the sample vessel is closed again. Closing the sample vessel is preferably carried out under hot steam, whereby there is obtained the advantage that the risk of contamination of the sample material in the sample vessel 4 in this step is reduced. Finally, the sample vessel 4 is removed from the internal chamber space 5. The method according to the invention has the advantage that it ensures a completely aseptic sampling, without the risk of contaminating the sample material or the material container.

In a preferred embodiment variant of the method, there is generated within the internal line space 14 of the sampling device 1 a negative pressure, and the sample material is conveyed into the internal line space 14, after the sample vessel 4 has been connected to the material container. Then there is generated a positive pressure within the internal line space 14, and the sample material is filled into the sample vessel 4. In this way there is obtained the advantage that the internal line space 14 is filled with sample material as completely as possible and that it is again emptied afterwards as completely as possible. In this way, the following cleaning steps will be facilitated, and it will be ensured that in each sampling the same amount of sample material is removed from the material container.

1. A sampling device for filling a sample vessel with sample material from a material container, wherein the sampling device comprises a first locking unit, a sterilization unit and a sampling chamber, wherein the sampling chamber has an internal chamber space, a manipulation unit, an opening unit arranged within the internal chamber space as well as a coupling unit arranged within the internal chamber space, the first locking unit is arranged between the material container and the coupling unit, and the sterilization unit is connected to the internal chamber space and configured to sterilize the internal chamber space, wherein the manipulation unit is configured to accommodate the sample vessel at least in part within the internal chamber space, movable between the opening unit and the coupling unit, the opening unit is configured to open and close the sample vessel, and the coupling unit is configured to be coupleable to the sample vessel.

2. A sampling device according to claim 1, wherein the coupling unit has sterilization openings and that the sterilization openings are connected to the sterilization unit.

3. A sampling device according to claim 1, wherein the sampling device has a first temperature sensor, which is arranged within the internal chamber space.

4. A sampling device according to a claim 1, wherein the sampling device has a sampling line having an internal line space which has a first end, at which the first locking unit is arranged, and second end, at which a second locking unit is arranged, wherein the second end of the sampling line is connected to the material container and the first locking unit and the second locking unit are configured to lock the sampling line.

5. A sampling device according to claim 4, wherein the internal line space has a volume, which determines a volume of sample material for filling the sample vessel.

6. A sampling device according to claim 4, wherein the sterilization unit is connected to the internal line space and configured to sterilize the internal line space.

7. A sampling device according to claim 4, wherein the sampling device has a second temperature sensor, which is arranged within the internal line space.

8. A sampling device according to claim 4, wherein the sampling device has an inert gas purging unit, which is connected to the internal line space and configured to generate a positive pressure within the internal line space.

9. A sampling device according to claim 4, wherein the sampling device has a negative pressure generating unit, which is connected to the internal line space and configured to generate a negative pressure within the internal line space.

10. A sampling device according claim 1, wherein the first locking unit has a passage cross-section of at least 30 mm and that the coupling unit is configured to be coupleable to a sample vessel, which has an opening area having a diameter of at least 30 mm.

11. A sampling device according to claim 4, wherein the sampling line, the first locking unit and the second locking unit have a passage cross-section of at least 30 mm and that the coupling unit is configured to be coupleable to a sample vessel, which has an opening area having a diameter of at least 30 mm.

12. A sampling device according to claim 1, wherein the sampling chamber has connecting elements, wherein the manipulation unit is accommodated slidable within the connecting elements

13. A sampling device according to claim 1, wherein the manipulation unit is configured as a slidable boundary surface of the sampling chamber, which has an opening adapted to a cross-section of the sample vessel.

14. A sampling device according to claim 1, wherein the manipulation unit has a handle.

15. A sampling device according to claim 1, wherein the sampling chamber has a holder arranged in the operational position underneath the manipulation unit.

16. A sampling device according to claim 15, wherein the holder is arranged in the operational position underneath the coupling unit.

17. A sampling device according to claim 1, wherein the sterilization unit is a steam generator.

18. A sampling device according to claim 17, wherein the steam generator generates steam of a temperature of at least 120° C.

19. A method for filling a sample vessel with sample material from a material container using a sampling device, comprising the steps of:
a) flooding an internal chamber space of the sampling device with hot steam of a temperature of at least 120° C.;
b) reducing the temperature of the hot steam to below 120° C.;
c) introducing the sample vessel into the internal chamber space;
d) opening the sample vessel;
e) connecting the sample vessel to the material container;
f) filling the sample vessel with sample material from the material container;
g) closing the sample vessel preferably under hot steam;
h) removing the sample vessel from the internal chamber space;

20. A method according to claim 19, comprising the steps of:
i) generating a negative pressure within an internal line space of the sampling device and conveying the sample material into the internal line space following step e);
j) generating a positive pressure within the internal line space following step i) and before step f).
